(19) United States
(12) Patent Application Publication
(10) Pub. No.: US 2006/0088943 A1
(43) Pub. Date: Apr. 27, 2006

(54) Method and apparatus for determining a concentration of a component in a mixture

(76) Inventors: Otto J. Prohaska, Beacon Falls, CT (US); Avinash Dalmia, Hamden, CT (US)

Correspondence Address: ST. ONGE STEWARD JOHNSTON & REENS, LLC, 986 BEDFORD STREET, STAMFORD, CT, 06905-5619, US

(21) Appl. No.: 11/265,731

(22) Filed: Nov. 2, 2005

Related U.S. Application Data
(63) Continuation-in-part of application No. 10/345,620, filed on Jan. 16, 2003.

(51) Int. Cl. G01N 33/00

(52) U.S. Cl. 436/155

(57) ABSTRACT

The invention relates to a method and apparatus for determining a concentration of a component in an unknown mixture. The method includes the steps of preparing a reactant having a specified pH level and a specified temperature and combining the unknown mixture with the reactant. The method further includes varying the pH level and temperature of the combination of the reactant and the unknown mixture to facilitate converting at least one selected component. Upon varying the pH level and temperature, the method will release volatiles from the selected component(s). Based on these released volatiles, which indicate the concentration of the selected component(s), the method detects the indication. The apparatus for determining a concentration of a component in an unknown mixture includes a container having a specified volume, a reactant chamber, and a sample chamber. The receptacle contains a reactant, placed within the reactant chamber, having a predetermined pH level and a predetermined volume. The receptacle also has a headspace sampling interface in contact with the container for permitting connection to a headspace sampling device and a sample introduction interface for permitting connection to a sample injector to introduce a sample into the sample chamber.

### CROSS-REFERENCE TO RELATED APPLICATIONS

This application is a continuation-in-part of co-pending U.S. patent application Ser. No. 10/345,620 for “Method and Apparatus for Determining a Concentration of a Component in a Mixture,” filed Jan. 16, 2003.

### FIELD OF THE INVENTION

The invention relates to a method and apparatus for determining an amount of a component present in a mixture.

### BACKGROUND OF THE INVENTION

Manners for detecting an amount of a desired component in an unknown mixture of components have evolved from simple mechanisms, such as chromatography and lithographs, to more complex and accurate mechanisms, such as sensors. Sensors are known to detect a concentration of a component introduced into the sensor.

However, false or inaccurate readings may occur if multiple gases are introduced across the sensor's sensing electrode because the sensor may sense gases not desired to be targeted by a user. Increasing the number of gases across the sensing electrode generally increases error. This problem may worsen when multiple gases having similar properties, such as chemical and/or electrical properties, come in contact with the sensing electrode, typically resulting in difficulty distinguishing a targeted component from other components having similar characteristics.

Additionally, inconsistencies in the testing environment may lead to repeatability problems, where a reading may not be confirmed by repeating the experiment without introducing additional deviation error. For example, a technician desiring to detect a selected gas at the sensing electrode may, during the experiment, need to mix a mixture of gases with a reactant in order to vaporize the selected gas. Measuring a precise amount of the reactant or varying the reactant's physical properties, in order to facilitate vaporizing the gas, often results in each reading being different from the next because repeatedly measuring a precise amount or repeatedly varying the physical properties in the same manners may prove difficult.

U.S. Pat. No. 6,143,246 to Lee et al. relates to an apparatus for measuring ammonia in wastewater. The invention discloses a method for adjusting the pH level of the sample to a predetermined level for a predetermined amount of time. The method further correlates the measurements of time and linear correlation constants in an inventive formula to arrive at a calculated concentration of ammonia. However, the reference is generally not applicable for detecting a component other than ammonia. The reference also does not typically relate to a method for detecting ammonia in a mixed solution of unknown chemicals.

U.S. Pat. No. 5,976,465 to Luzzana et al. generally relates to a method for determining a concentration of a sample by measuring pH at the beginning and end of a reaction of the sample with a reactant. The change in pH is indicative of the sample concentration. Regulating temperature and minimizing the effects of temperature on pH is disclosed. However, the reference does not typically determine the concentration by measuring the sample directly. Instead, the reference normally measures changes in the pH level of the solution, the change in pH being indicative of the sample concentration. This indirect measurement of the sample concentration may introduce error into the readings because the resulting differences in pH would likely entail converting the pH difference to a concentration measurement. Furthermore, the reference does not typically address or reduce the likelihood of having undesired components participating in the reaction and interfering with the desired component's measurement.

U.S. Pat. No. 5,991,020 to Loge relates to a method for determining a concentration of atomic species in gases and solids. The method requires measuring at least two emission intensities from a species in a plasma containing the species after a sufficient time interval and plasma has had an opportunity to be generated. Concentration is then derived from emission intensities of the desired species in the sample. Similar to Luzzana, this reference often measures concentration indirectly. The concentration is typically derived from measured intensities and it is this extra step of derivation, a step obviated in direct measurements of the sample concentration, that may cause error in readings. Furthermore, the reference does not typically address or reduce the likelihood of having undesired components participating in the reaction and interfering with the desired component's measurement.

No reference or combination of references discloses a method for determining a concentration of a component dissolved in a mixture of components by directly measuring the component. Additionally, no reference reduces a likelihood of having undesired components participating in the reaction and interfering with the desired component's measurement. Furthermore, no reference discloses a simple and easy-to-use device for enhancing repeatability readings by reducing experimental or human error during experiments.

What is desired, therefore, is a method for determining a concentration of a component dissolved in a mixture of components. What is also desired is a method of determining the concentration by directly measuring the selected component. A further desire is to reduce a likelihood of having undesired components participating in the reaction and interfering with the desired component's measurement. A still further desire is to provide a device that is simple and easy to use that enhances repeatability readings and reduces experimental error.

### SUMMARY OF THE INVENTION

Accordingly, it is an object of the invention to provide a method for determining a concentration of a component in an unknown mixture.

Another object of the invention is to provide a method for substantially transforming a selected component, originally combined in the unknown mixture, into a gaseous phase.

A further object of the invention is to provide a method for inhibiting unselected components of the mixture from transforming to a gaseous phase and interfering with detection of the selected component.

Still another object of the invention is to provide a device that is simple and easy to use to enhance repeatability readings.

Yet another object of the invention is to provide a device that reduces experimental error.

These and other objects of the invention are achieved by provision of a method for determining a concentration of a component in an unknown mixture of components. The method includes the steps of preparing a reactant having a specified pH level and a specified volume and combining the unknown mixture with the reactant. The method further includes varying the pH level and temperature of the combination of the reactant and the unknown mixture to facilitate converting at least one selected component. Upon varying the pH level and temperature, the method will release volatiles from the selected component(s). The method then detects these released volatiles, which indicate the concentration of the selected component(s).

The method further includes the step of calculating the concentration of the component(s) in the unknown mixture based on the detected volatiles, or indication. Prior to detecting the indication of the concentration of the selected component(s), the method transforms the selected component(s) to a gaseous phase.

In conjunction with varying the pH level and temperature of the combination of the reactant and the unknown mixture, the method may include determining a dissociation constant of the selected component and adjusting the pH level of the combination relative to the dissociation constant to facilitate releasing volatiles from a desired component and/or suppressing the release of volatiles from undesired components.

In another aspect of the invention, a receptacle is provided for determining a concentration of a component in an unknown mixture. The receptacle includes a container having a specified volume, a reactant chamber, and a sample chamber. The receptacle contains a reactant, placed within the reactant chamber, having a predetermined pH level and a predetermined volume. The receptacle also has a headspace sampling interface in contact with the container for permitting connection to a headspace sampling device and a sample introduction interface for permitting connection to a sample injector, which introduces a sample into the sample chamber. Optionally, the sample introduction interface may be coupled to a valve for permitting a fixed amount or volume of the sample to enter the container.

The receptacle further includes the unknown mixture placed in the sample chamber. In certain embodiments, the receptacle includes a mixer in contact with the container for mixing the reactant and sample.

The receptacle also includes a separable mechanism for separating the reactant chamber from the sample chamber. The separable mechanism is removable or has a portion that is removable so that the reactant and sample may be combined.

In further embodiments, the receptacle includes a second reactant placed in a second reactant chamber for further combination with the first reactant and mixture. In these embodiments, there is also a separable membrane separating the reactant chambers and mixture.

In another aspect of the invention, the apparatus for determining a component in an unknown mixture further includes, in addition to the receptacle described above, a heating element in contact with the container for heating the contents of the container. The apparatus also includes a timer for setting a heating time for the heating element, a headspace sampling device coupled to the headspace sampling interface, and an electronic circuit in contact with, and for actuating, the heating element, the timer, and the headspace sampling device.

The headspace sampling device may include an electrochemical gas sensor for sensing volatile releases in the container.

It should be understood that the apparatus is capable of receiving any one of a plurality of containers of varying sizes and having varying volumes of reactants with varying pH levels. To this end, the apparatus includes a receiver to accommodate any one of the plurality of containers.

The invention and its particular features and advantages will become more apparent from the following detailed description considered with reference to the accompanying drawings.

### BRIEF DESCRIPTION OF THE DRAWINGS

FIG. 1 depicts a method for determining a concentration of a component in an unknown mixture in accordance with the invention.

FIG. 2 more particularly depicts the conversion and suppression steps of the method shown in FIG. 1.

FIG. 3 depicts an apparatus for practicing the method shown in FIG. 1.

FIG. 4 depicts further features of the apparatus shown in FIG. 3 and for practicing the method shown in FIG. 1.

FIG. 5 depicts a table of ECS and GC/SCD measurements while varying acid concentrations and temperatures.

FIG. 6 depicts another table of ECS and GC/SCD measurements while varying acid concentrations and temperatures.

FIG. 7 depicts a table of GC/SCD and ECS responses.

FIG. 8 depicts a table of GC/SCD measurements.

FIGS. 9-15 depict chromatograms for the sugar samples during different experiments.

FIG. 16 depicts a graph of sensor response over time for sugar sample 3.

FIGS. 17a-17b depict a graph of sensor response versus H₂S concentration.

FIGS. 18-29 depict various correlations between the area counts of H₂S, SO₂, and COS versus ECS responses while varying acid concentrations and temperatures

### DETAILED DESCRIPTION OF THE DRAWINGS

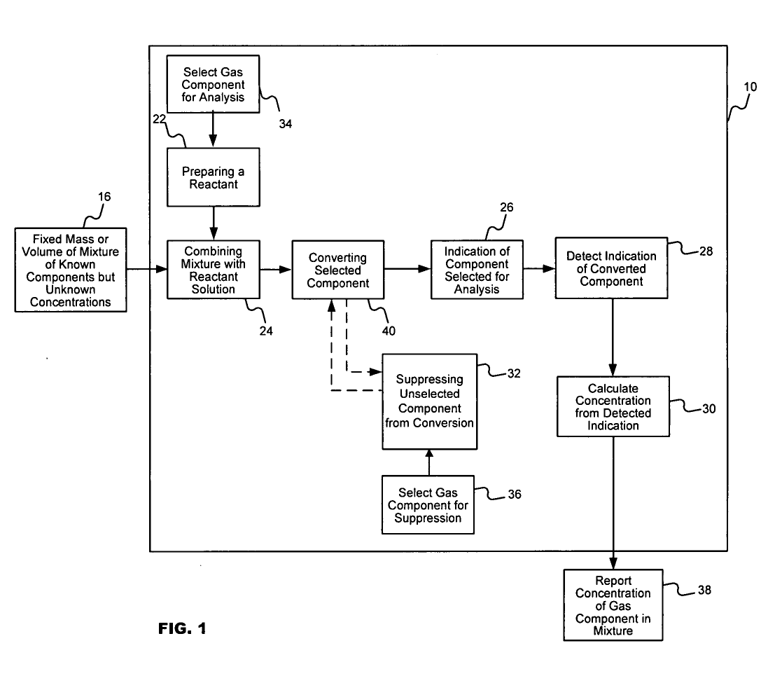

FIG. 1 depicts the method 10 for determining a concentration of a component in an unknown mixture in accordance with the invention. Method 10 determines the concentration of a component in a liquid or solid phase by transforming the component to a gaseous phase. Once in the gaseous phase, the component is detectable by a detection unit, such as an electrochemical gas sensor or other unit for detecting vapors. Method 10 further includes steps for enhancing conversion of the selected, or desired, component 34 and steps for suppressing, or inhibiting, the conversion of unselected components.

As shown in FIG. 1, method 10 includes the step of preparing 32 a reactant having a specified pH level, a specified volume, and, optionally, a specified temperature and then combining 24, or mixing, the prepared reactant with a mixture 16 of known components. Although method 10 would properly function if the pH level, temperature, or volume of the reactant were not known, eliminating as many variables from method 10 increases the likelihood of yielding an accurate concentration determination of selected component 34. Mixture 16 contains, among other components, the component 34 to be selected for determining its concentration.

In the preferred embodiment, although mixture 16 contains various components, an operator using method 10 should know the general total volume of mixture 16. Similar to the reasons for knowing the volume of the reactant, knowing the volume of mixture 16 reduces the number of variables for which to solve, thereby yielding a more accurate concentration determination. In other embodiments, method 10 may be practiced with an unknown volume of mixture 16. However, in these embodiments, accuracy may be compromised.

Because the volume of mixture 16, volume of the prepared reactant, as well as the pH level and temperature of the reactant, are within the control of the operator, the operator may eliminate these variables.

Further, an operator using method 10 should also determine, or select, the component 34 for analysis in which its concentration is determined. Moreover, although mixture 16 contains numerous components, the operator need not know the identity of all of the components. The operator needs to know that selected component 34 is in mixture 16, albeit in the liquid or solid phase.

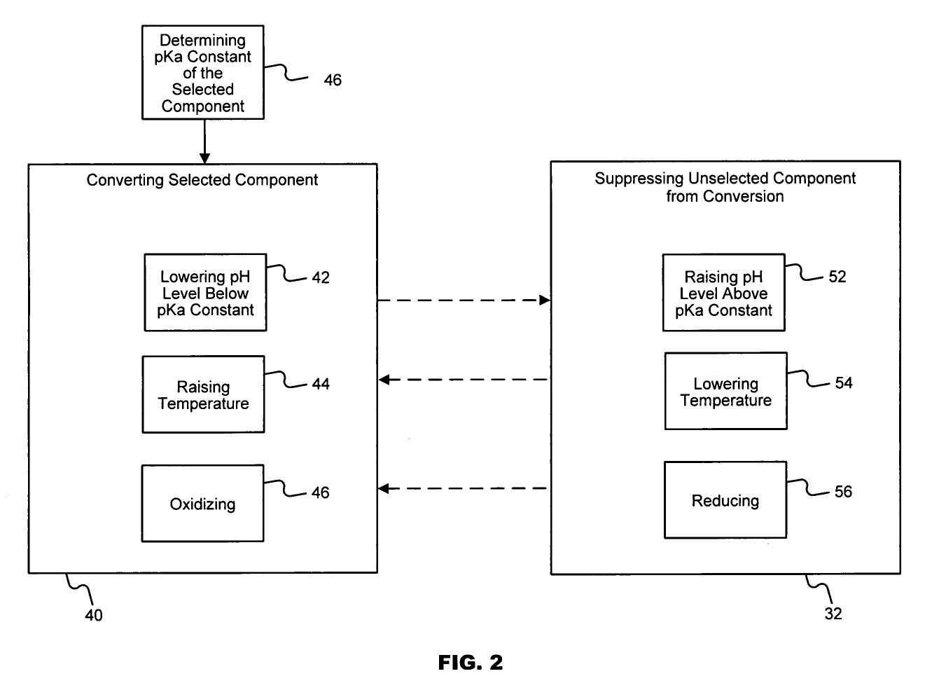

To determine the concentration of selected component 34, method 10 converts 40 selected component 34, or transforms component 34 to a gaseous phase. Converting 40 selected component 34 is particularly important because the more efficiently selected component 34 is converted, the more accurately the concentration may be determined. Efficient conversion is defined to be transforming a substantial percentage of selected component 34 from a liquid or solid phase to a gaseous phase. Transforming 100% of selected component 34 is ideal but not required for method 10 to properly function. The more efficiently, or closer to 100%, selected component 34 is converted, or transformed to a gaseous phase, the greater the amount of gas created and the more volatiles are released, which is representative of the amount, or concentration, of selected component 34. This leads to a more accurate concentration determination, whereas transforming a small amount of selected component 34 to a gas may lead to a lower concentration determination than is present in mixture 16. Similarly with knowing the volume of mixture 16 and other variables related to the prepared reactant, efficiently converting 40 selected component 34 improves the likelihood of a more accurate concentration determination. The steps of converting 40 selected component 34 and suppressing 32 unselected components will be more particularly described under FIG. 2.

After selected component 34, or the indication 26 of a component selected for analysis, has been converted 40, volatiles are automatically released from selected component 34, which is now in the gaseous phase. Volatiles are defined to be contaminants, bacteria, or any kind of releases indicative of selected component 34. It is these volatiles, or indications 26 of selected component 34, that are subsequently detected by the detection unit, such as an electrochemical gas sensor or other unit for detecting vapors. Hence, detecting a concentration of selected component 34 is performed by detecting 28 indications of selected component 34, such as the volatile releases.

Method 10 further includes calculating 30 the concentration of selected component 34 and reporting 38 the concentration. Calculating 30 the concentration is performed using correlation information, such as the following formula, to correlate the amount of indications 26, or volatiles, detected by the detection unit and the amount, or concentration, of selected component 34 originally in mixture 16.

The liquid and gas phases of component 34 may be expressed in the following equation:
pKa=pH+Log₁₀ ((Component in gas phase/Component in liquid phase)) formula 1

where pKa is a known constant, pH of the Combination is measured, gas phase of component 34 is also measured, or detected 28, and the liquid phase of component 34 is to be solved.

Based on the above formula 1, and solving for the component is the gaseous phase, we find that if pKa−pH results in a number less than zero, then the component in the liquid phase is greater in concentration than the component in the gas phase, or the liquid has a concentration ratio greater than gas. If pKa−pH results in a number greater than zero, then the component in the gaseous phase is greater in concentration than the component in the liquid phase, or the gas has a concentration ratio greater than liquid.

In further embodiments where pKa−pH results in a number less than −1, then the component in the liquid phas is dominant, or the liquid has a concentration at least 10 times greater than the concentration of gas. If pKa−pH results in a number greater than 1, then the component in the gaseous phase is dominant, or the gas has a concentration at least 10 times greater than the concentration of liquid. Because the gaseous component is to be detected, it is preferred that the gaseous phase be dominant over the liquid phase.

By lowering the pH level of the Combination below the pKa constant, selected component 34 is more likely to vaporize and, specifically, more likely to efficiently vaporize because the result of pKa−pH is greater than zero.

Reporting 38 the concentration is performed through all known or novel manners for reporting information, such as merely displaying the concentration on a monitor or LCD. Reporting 38 may also be storing or sending the concentration to a computer or other storage device. Reporting 38 is not germane to the invention and should not be a limitation of method 10.

FIG. 2 more particularly depicts the steps for converting 40 selected component 34 and suppressing 32, or inhibiting, unselected components from conversion.

After the desired component has been selected 34 for analysis by an operator, converting 40 selected component 34 entails practicing steps to facilitate transformation of selected component 34 from a liquid or solid phase to a gaseous phase. Converting 40 includes determining a disassociation constant (“pKa constant”) of selected component 34 and adjusting the pH level of the combination of the reactant and mixture 16 (“Combination”) relative to the pKa constant, which is an indication of the component's ability to partition between liquid and gas phases.

In the embodiment shown in FIG. 2, lowering 42 the pH level is one of several steps that facilitate converting 40 selected component 34. However, lowering 42 the pH level is not universally applicable to convert 40 all selected components. In other embodiments, not shown, raising the pH may facilitate converting 40 selected component 34. The raising or lowering of the Combination's pH level for facilitating converting 40 selected component 34 depends on the type of component selected for analysis and the mixture in which the component is placed.

Additionally, converting 40 includes varying a temperature of the Combination. For example, when practicing method 10 for converting the selected component, such as H₂S, the temperature is typically raised to between approximately 50° C. and 80° C. and, preferably, approximately 80° C. However, this 80° C. temperature is merely an example and may vary to convert different components or compounds from different mixtures 16. Furthermore, this temperature was empirically determined for converting SO₂ and later experiments above or below 80° C. may be used with respect to converting SO₂.

In the embodiment shown in FIG. 2, raising 44 the temperature is another step that facilitates converting 40 selected component 34. However, raising 44 the temperature is not universally applicable to convert 40 all selected components. In other embodiments, not shown, lowering the temperature may facilitate converting 40 selected component 34. The raising or lowering of the Combination's temperature for facilitating converting 40 selected component 34 depends on the type of component selected for analysis and the mixture in which the component is placed.

Further, by increasing the temperature, undesired interferences may be suppressed, which facilitates detection of desired components. For example, SO₂, which may interfere with the detection of H₂S, is converted to SO₃ at higher temperatures, such as 80° C. SO₃ is not active, or does not provide an electrochemical signal that may interfere with the detection of H₂S and, hence, the detection of H₂S is facilitated. Suppression is optional and need not required for complete conversion 40 of selected component 34.

Additionally, as shown in FIG. 2, converting 40 selected component 34 may also include oxidizing or reducing the Combination. Oxidation and reduction include all known or novel procedures in the art for oxidizing or reducing the Combination.

In the embodiment shown in FIG. 2, oxidizing 46 the Combination is another step that facilitates converting 40 selected component 34. However, oxidizing 46 the Combination is not universally applicable to convert 40 all selected components. In other embodiments, not shown, reducing the pH may facilitate converting 40 selected component 34. Whether to oxidize or reduce the Combination for facilitating converting 40 selected component 34 depends on the type of component selected for analysis and the mixture in which the component is placed.

In further embodiments, selected component 34 may be a compound that efficiently transforms to a gaseous phase without adjusting the pH level or temperature of the Combination or without oxidation or reduction. Hence, selected component 34 is efficiently converted due to the chemical properties of selected component 34 among the other compounds in mixture 16 and/or the reactant.

In some instances, converting 40 selected component 34 may cause other, unselected components to also convert. This is because converting entails subjecting the Combination of both the reactant and mixture 16 to the same temperature and/or pH adjustments. For components having similar chemical properties as selected component 34, these components may be inadvertently converted along with selected component 34. In cases where conversion affects unselected components, suppression in addition to or instead of conversion may remedy the problem of inadvertently converting unselected components.

Suppressing 32 unselected components inhibits the unselected components from conversion. Suppressing 32 includes adjusting the pH level of the Combination relative to the pKa constant. For the example shown in FIG. 2, unselected components may be suppressed by raising 52 the pH level above the pKa constant and lowering 54 the temperature of the Combination.

Similar to the step for converting 40 selected component 34, the degree of raising 52 the pH level or lowering 54 the temperature varies according to the type of selected component 34 and mixture 16 in which selected component 34 is placed. Further, depending on these factors, the temperature may be raised in order to suppress 32 unselected components.

Additionally, the degree of reducing 56 the Combination for facilitating suppression 32 of unselected components from being converted varies according to the type of selected component 34 and mixture 16 in which selected component 34 is placed. Further, depending on these factors, the Combination may be oxidized in order to suppress 32 unselected components.

As shown in FIG. 2, the Combination cannot have its pH level lowered below the pKa constant to facilitate converting 40 selected component 34 at the same time the pH level is raised above the pKa constant to suppress unselected components. However, these steps may be performed in sequence one after the other or spaced apart after a time interval. Further, the Combination's pH may be adjusted simultaneously or sequentially with the temperature for facilitating conversion and suppression. The Combination may also be oxidized and reduced independently from adjusting the pH and temperature.

It should be understood that converting 40 selected component 34 and/or suppressing 32 an unselected component does not require any of the above steps of raising 44 or lowering 54 the temperature and lowering 42 or raising 52 the pH level of the Combination relative to the pKa constant. Oxidation or reduction may also not be required for converting 40 selected component 34. Converting 40 or suppressing 32 may entail practicing one, several, all, or some combination of these steps. The steps method 10 practices for converting 40 and/or suppressing 32 depends upon the type of selected component 34 and mixture 16 in which selected component 34 is placed.

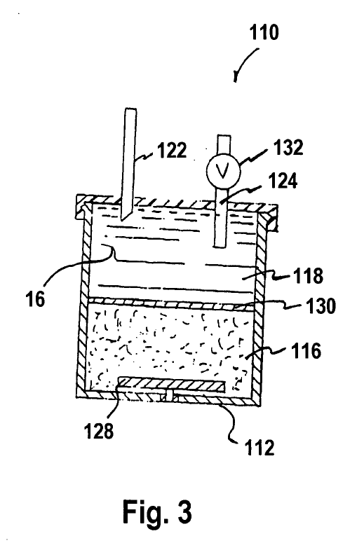

FIG. 3 depicts the apparatus 100 for determining a component in an unknown mixture in accordance with the invention. Sample preparation receptacle 110 provides a reactant having a specified volume and specified pH level, among other known properties, such as density, mass, temperature, and the like. Sample preparation receptacle 110 aides an operator in practicing method 10, particularly step one of method 10 embodied in FIG. 1 for preparing a reactant having a specified pH level and a specified volume. By having a predetermined pH and volume, receptacle 110 reduces experimental error that may be introduced if the operator were to measure pH and volume of the reactant, especially if the experiment required this be done with particular precision or if the experiment were repeated.

Receptacle 110 includes a container 112 having a specified volume of containment, wherein container 112 further includes a reactant 116 chamber for placing a reactant and a sample 118 chamber for placing a sample, or mixture 16, within container 112. The reactant may be a liquid, solid, or gas. Depending on the type of component selected for conversion or mixture 16, the reactant's phase may vary.

Container 112 further includes a headspace sampling 122 interface for coupling a detection unit, such as a headspace sampling device, to container 112 for detecting the volatiles released from the converted selected component 34. Another detection unit may be a sensor, electrochemical gas sensor, or any unit capable of detecting volatile releases from the converted selected component 34.

Container 112 further includes a sample introduction 124 interface for providing an inlet for mixture 16, or the sample to be analyzed, to enter container 112 and, more specifically, enter sample chamber 118. To facilitate introducing a specific amount or volume of mixture 16 into container 112, valve 132 is provided in cooperation with sample introduction 124 interface.

Both headspace sampling 122 and sample introduction 124 interfaces are merely ports or connections and may have the same limitations. The design of these interfaces or manners for coupling with the detection unit or source for introducing mixture 16 should not be a limitation of receptacle 110.

Receptacle 110 may further include a mixer 128 in contact with container 112 for mixing the reactant and mixture 16 together once both the reactant and mixture 16 have been placed in their respective chambers. Mixer 128 may be internal, as shown in FIG. 3, or external of container 112 and includes all known or novel mixers for mixing liquids or solids or both. Mixer 128 may also be inserted into container 112.

In addition to or instead of mixer 128, receptacle 110 may further include a separable mechanism 130, such as a membrane, for separating reactant 116 chamber from sample 118 chamber. Separable mechanism 130 may be removable or have a portion of it that is removable so that mixture 16 and the reactant may be combined. Moreover, in certain embodiments, separable mechanism 130 may be automatically dissolvable overtime once mixture 16 has been added to sample 118 chamber. This automatic dissolution may be due to the chemical reaction between separable mechanism 130 and the reactant or mixture 16. In further embodiments, separable mechanism 130 is porous or has apertures for permitting the reactant and mixture 16 to mix.

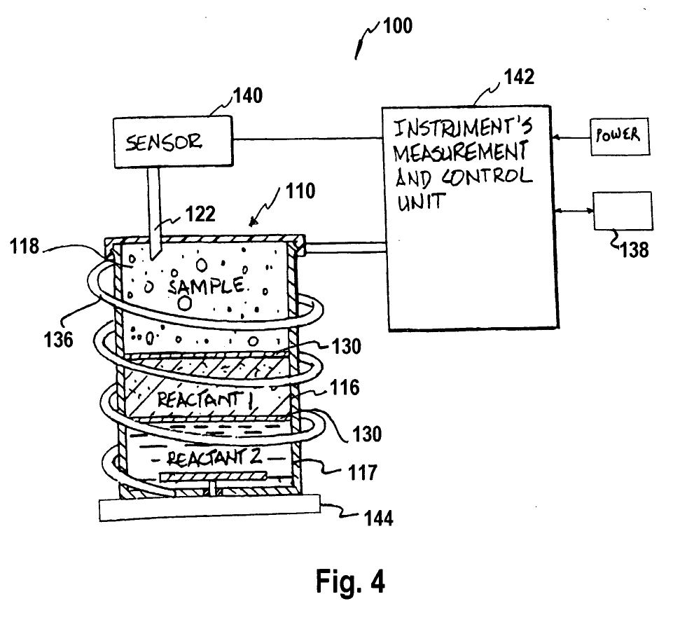

In further embodiments, receptacle 110 includes more than one reactant chamber. As shown in FIG. 4, a second reactant 117 chamber is used. Separable 130 mechanism separating reactant 116 chamber from second reactant 117 chamber includes all of the limitations described above. In addition, the order of sample 118 chamber, reactant 116 chamber, second reactant 117 chamber, or any additional reactant chamber is not to be a limitation on the invention. Also, the order in which separable mechanism 130 is removed or dissolved is not a limitation on the invention.

In addition to receptacle 110 and shown more particularly in FIG. 4, apparatus 100 includes heating element 136 in contact with container 112 for heating the contents of container 112, timer 138 for setting a heating time for heating element 136, headspace sampling device 140 coupled to headspace sampling 122 interface for measuring volatile releases from mixture 16, and electronic circuit 142 in connection with heating element 136, headspace sampling device 140, and timer 138 for actuating and giving power for these items to function properly.

Heating element 136 is any heat conducting device for heating receptacle 110. Preferably, heating element 136 wraps about receptacle to heat the contents of receptacle 110 evenly. Desirably, heating element 136 should be adjustable such that when heating element 136 is coupled to electronic circuit 142, an operator operating electronic circuit 142 may vary the heat intensity or power of heating element 136. In some embodiments, heating device 136 is a heating coil. Heating element 136 may also have an automatic shut off/turn on switch to maintain a desired temperature.

Headspace sampling device 140 is any detection unit capable of detecting volatiles indicative of selected component 34, such as an electrochemical gas sensor or other unit for detecting vapors.

Electronic circuit 142 is an electrical connection to power heating element 136, timer 138, and headspace sampling device 140. Electronic circuit 142 may also include controls for manipulating the amount of power to, as well as adjusting the operation of, each of these items. For example, electronic circuit 142 facilitates setting timer 138, operating headspace sampling device 140, or varying a temperature or intensity of heating element 136. In certain embodiments, electronic circuit 142 performs what otherwise would be manually laborious, tedious, or time consuming operations and centralizes the operations in an electrical panel having controls for each of the above mentioned items.

Apparatus 100 may further include receiver 144 for receiving any one of a plurality of receptacles 110, where receptacles 110 vary in size, geometry, or weight. Receiver 144 may be a platform for receiving and supporting any container 112 as well as heating element 136.

Although the invention has been described with reference to a particular arrangement of parts, features an the like, these are not intended to exhaust all possible arrangements or features, and indeed many other modifications and variation will be ascertainable to those of skill in the art and from the following example.

### EXAMPLE

Experimental Data

In an experiment where 50% sugar and 50% acid solution are mixed together, a sample of gas from a headspace (the area immediately above the mixture) is taken and injected into a GC column to separate the sulfur compounds. Four different grades of sugars were used for measurements. To make pH 1 solution, 0.1 M Phosphoric acid solution is added to sugar. To make pH 3 solution, 0.001 M Phosphoric acid solution is added to sugar. After separation of H₂S and SO₂ in a GC column, the sulfur compounds were measured using a SCD (Sulfur Chemiluminescence Detector). At room temperature, and by changing the pH of the mixture without changing the temperature, it was observed that the peak area of SO₂ was suppressed (stays in liquid phase) by a factor of between approximately 150 and approximately 400 by changing the pH of the acid solution (the 50% acid solution and not the entire mixture) from 1 to 3. See attached table 1, lines 1-10 for before and after peak areas of SO₂ and H₂S for specific measurements.

During this same experiment, the peak area of H₂S was suppressed by a factor of between approximately 2 and approximately 6 (by changing the pH of the acid solution from 1 to 3). See table 1, lines 1-10.

As the experimental data shows, although both H₂S and SO₂ are suppressed, the SO₂ was suppressed by a much larger degree than H₂S, which agrees with the application that one compound is suppressed in order to make the other compound more detectable.

This experiment also shows that by changing the pH to a value above the pKa of SO₂ (1.85), the resulting suppression of SO₂ is to a larger degree than the suppression of H₂S (pKa of 7.05). This can be explained further by the following equation:

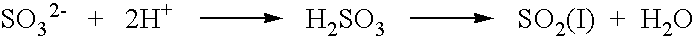

The concentration of SO₂ in liquid and gas phase is correlated by an equilibrium constant.

From the above equation, it can be seen that the lower pH leads to higher concentration of hydrogen ions and evolution of SO₂ from sulfite present in sugar.

The GC-SCD experiments were conducted to show that major sulfur component in headspace is SO₂ when pH of acid solution is 1 and the major component is H₂S when pH of acid solution is 3. It was observed that the response of electrochemical sensor correlated with SO₂ peak area from GC-SCD measurements when pH of solution was 1 and it correlated with H₂S peak area when pH of solution was 3.

In another experiment where temperature was changed from approximately 25° C. to approximately 80° C. but where the pH was held constant at 1, it was observed that the peak area of H₂S increased by a factor of between approximately 2 and approximately 6 but the peak area for SO₂ was suppressed by a factor of between approximately 150 and approximately 400. Similar to the above result, SO₂ is suppressed to a larger degree than H₂S. See table 1, lines 1-5 and 26-30.

Although one could expect H₂S to have also been suppressed but to a lower degree than SO₂, the fact that H₂S increased rather than be suppressed may have been due to presence of a sulfur compound in the sugars which released H₂S at higher temperatures. This is evidenced by the fact that all of the sugars showed an increase in H₂S as opposed to some of the sugars.

However, in theory and without interference, H₂S would have been suppressed as well but by a factor far less than SO₂, which was between approximately 150 and approximately 400.

Theoretical Data that Correlates with the Experimental Data

From the CRC Handbook of chemistry and physics, the solubility of SO₂ in a liquid phase is about 0.0246 mole fraction SO₂ in liquid phase at 1 atm. Converting this mole fraction to kg/m³ we arrive at 87.4 kg/m³.

The mole fraction of SO₂ in gas phase at 1 atm in kg/m³ is 2.626 kg/m³. Therefore, K=87.4/2.616=33.4

In reference to the below formula and as explained below, by choosing K=1, Applicants are using a worst case scenario since K is normally always greater than 1 (see experimental and theoretical data) since K=1 means log1=0. If K were greater than 1, pH would be greater in order to solve for the ratio of gas phase/liquid phase. The larger the K, the smaller this ratio becomes and this means the suppression is more efficient. Hence, assuming K=1 increases this ratio. Therefore, if solving for the ratio when K=1 provides satisfactory suppression (e.g. the ratio is equal to or less than 0.1), then larger K would provide better suppression.

Now we use the formula stated in the previous Response and in the application.
pKa=pH+logK+log (gas phase/liquid phase)
1.85=pH+log1+log (gas phase/liquid phase)
1.85=pH+0+log (gas phase/liquid phase)

Next we choose a pH at least one greater than the value of the pKa of the compound we wish to suppress, which is SO₂. The pH must be below the value of the pKa of the compound we do not wish to suppress. Hence, in this example, the pH should be between 2.85 and 7.05.
1.85=2.85+log (gas phase/liquid phase)
−1=log (gas phase/liquid phase)

Take the antilog of both sides to arrive at 0.1=gas phase/liquid phase, meaning 90% suppression of SO₂.

In the event the value of pH that is chosen is greater than 1 above the pKa, it would result in better efficiency.
1.85=3.85+log (gas phase/liquid phase)
0.01=gas phase/liquid phase or 99% suppression of SO₂.

If pH is less than 1 above the pKa, it would result in less than 90% suppression.

If K is greater than 1, this merely increases the numerical values on the right hand side of the equation and increases suppression. Hence, choosing K=1 and pH being 1 above the pKa are for the worst case calculation of the suppression of SO₂.

Comparison of ECS and GC Results for Sugar Quality Control Applications

Goals

The main goal of this example is to demonstrate the correlation of H₂S measurements performed by a gas chromatograph (GC) and by an electrochemical sensor (ECS) device.

A second goal of this example is to determine the impact of sample preparation parameter variations on the GC and ECS measurements for the validation of an objective ECS-based sugar QC procedure.

Summary

Demonstrating the correlation of H₂S measurements preformed by GC and ECS devices was accomplished by simultaneously supplying both instruments with H₂S/air mixes, where H₂S concentrations were between approximately 0 and approximately 500 ppb. In addition, it was shown that an ECS-based QC instrument for sugar is feasible, using sugar/acid solutions heated at a certain temperature for a certain time. A QC instrument might also be feasible under certain sugar/acid solution preparation conditions without heating the sample; such a device would allow fast QC turn-around times due to instant measurements.

Experimental Procedure

Instrument Conditions:

For the GC measurements, a headspace sampler and a sulfur chemiluminescence detector (GC/SCD) were used. For the GC measurements, a wide bore capillary column (DB-1, 5μ, 60 m, 0.53 mm ID) was used and maintained isothermal at 30° C. The headspace system was maintained at 30° C. and the transfer line was maintained at 35° C. The ECS measurements were performed with the electrochemical sensors. From the vial the sample was injected simultaneously into the GC column and the ECS, which were connected in parallel via a Y junction piece. The injection flow rates for ECS and GC columns were 5 and 10 cc/min, respectively. The injection time was 0.08 min. In addition, experiments were conducted in which a 2.5 ml syringe was used to inject the headspace samples from vials into electrochemical sensors from pure acid solutions and sugar/acid solutions at different temperatures and acid concentrations.

Calibration Standards:

Gas standards of different concentrations of H₂S were prepared for the establishment of the calibration curve by diluting an approximately 2 ppm H₂S/air gas mix using high-precision mass flow controllers. Similarly, the standards of gas mixes of 5 ppm COS/100 ppb H₂S/air and 1 ppm SO₂/air were measured with both the electrochemical sensor and the GC/SCD instrument.

Sample Preparation:

Four sugar samples, 1-4, provided by an outside vendor. The experiments were conducted with blank 5 ml phosphoric acid solutions (reagent grade from Aldrich) of different concentrations (0.1M. and 0.001M), and 5 gm of sugar mixed with 5 ml acid solution of these two concentrations in 22 ml headspace vials (sugar/acid solutions). The sugar/acid solutions were mixed and maintained for 30-60 minutes at room temperature (or 20-27° C.), 50° C., and 80° C. The high temperature solutions were cooled down to room temperature. These experimental conditions were selected to explore the possibility of performing the measurements in a shorter period of time or without heating the sample.

Results

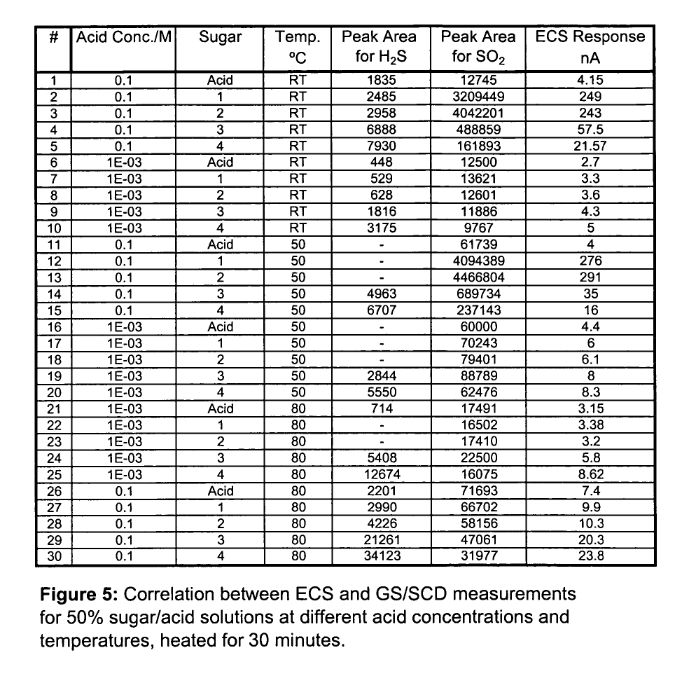

FIG. 5 displays the ECS and GC/SCD results obtained simultaneously from sugar/acid solutions under varying acid concentrations and temperature conditions.

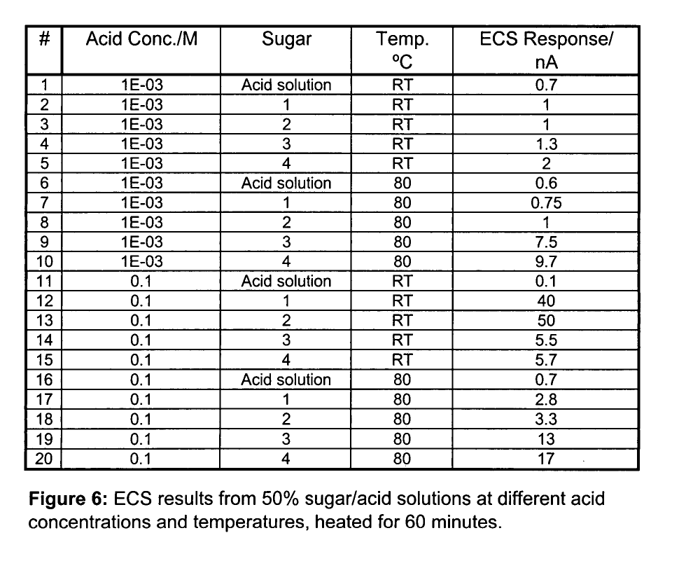

FIG. 6 summarizes the ECS measurements, obtained from the headspace of sugar/acid solutions under varying acid concentrations and temperature conditions, where the sample gas was transferred from the vial to the ECS device. FIG. 6 depicts a similar experiment as FIG. 5 depicts but with a 60 minute experiment time as opposed to 30 minutes.

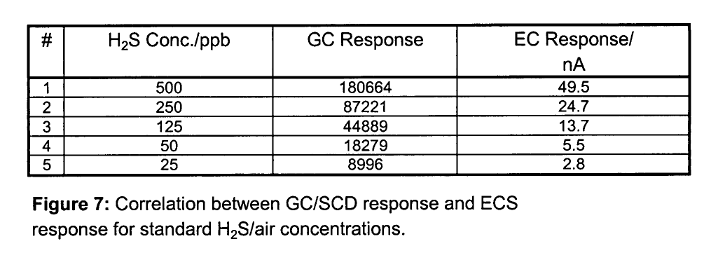

FIG. 7 shows the correlation between electrochemical responses and GC/SCD results for H₂S/air gas standards of H₂S concentrations between approximately 0 and approximately 500 ppb. This experiment determines the quantity of H₂S present in the sugar samples listed in FIG. 7 based on ECS Response.

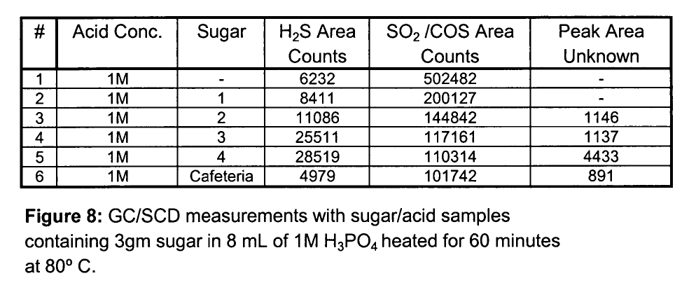

FIG. 8 shows the GC/SCD measurements under sample preparation conditions. pH in this experiment was approximately equal to 0.

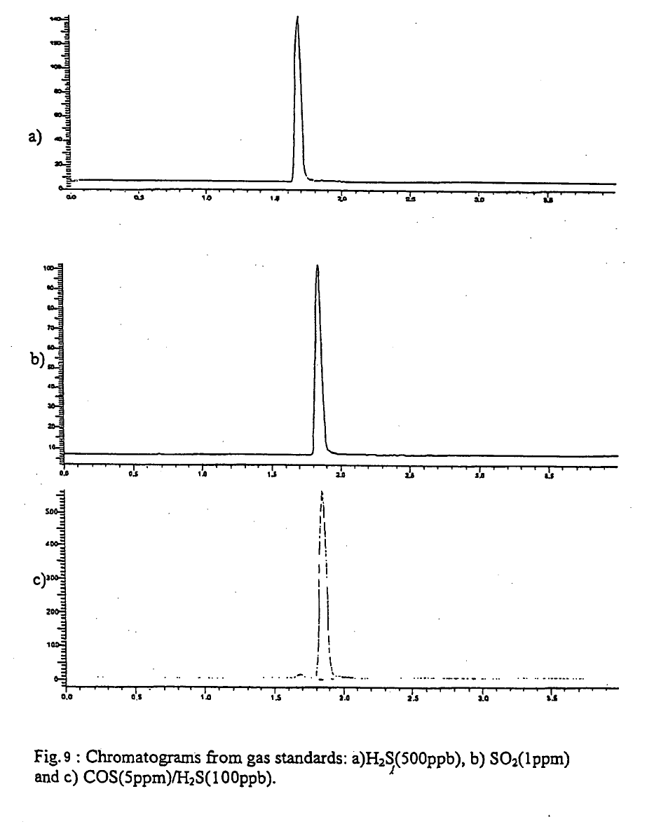

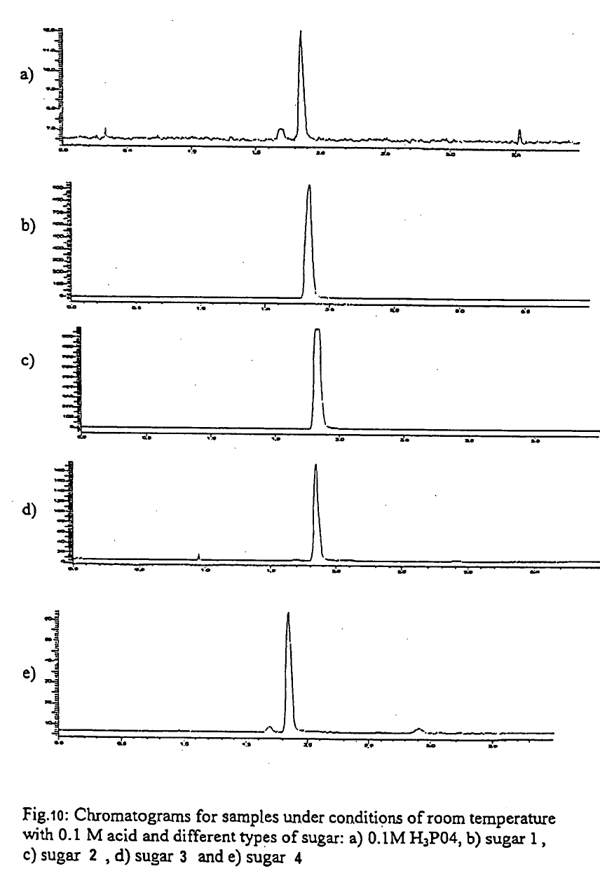

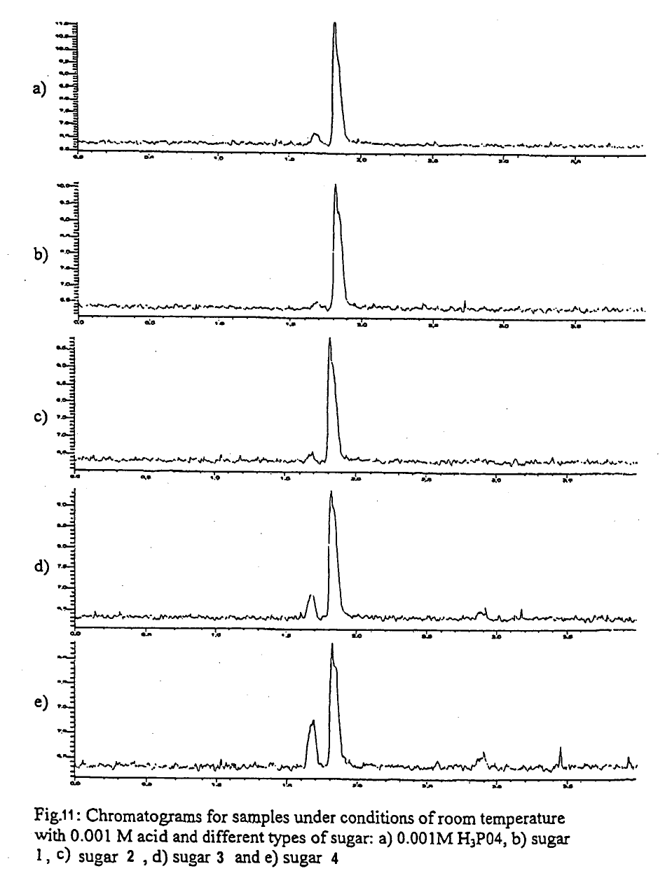

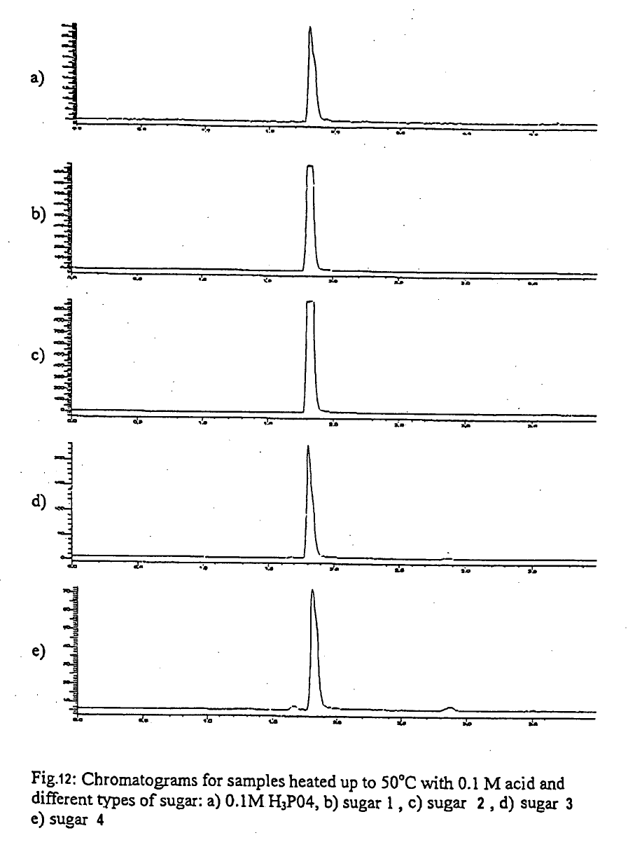

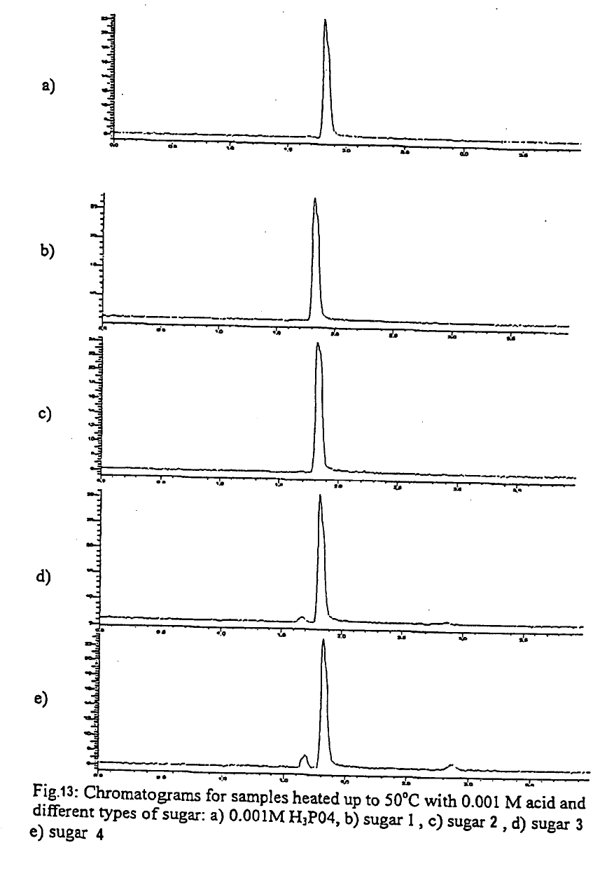

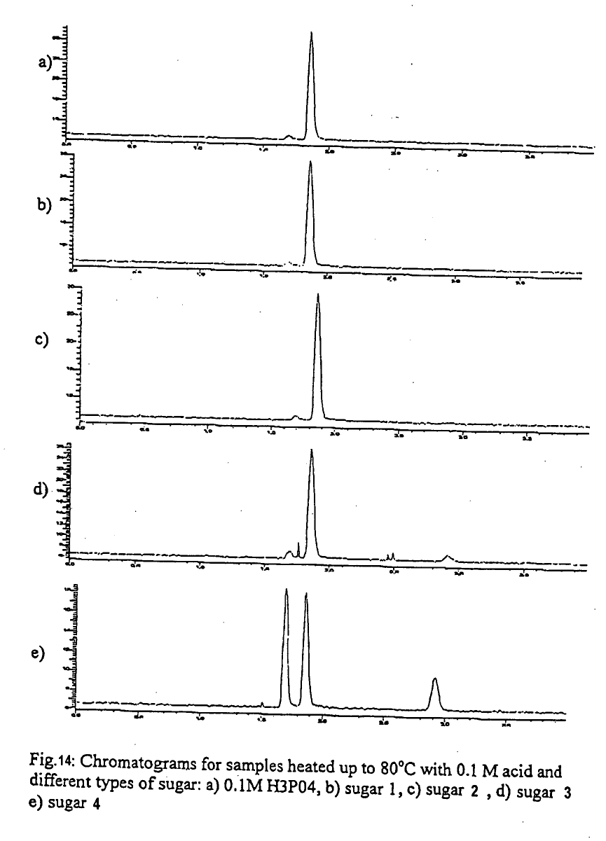

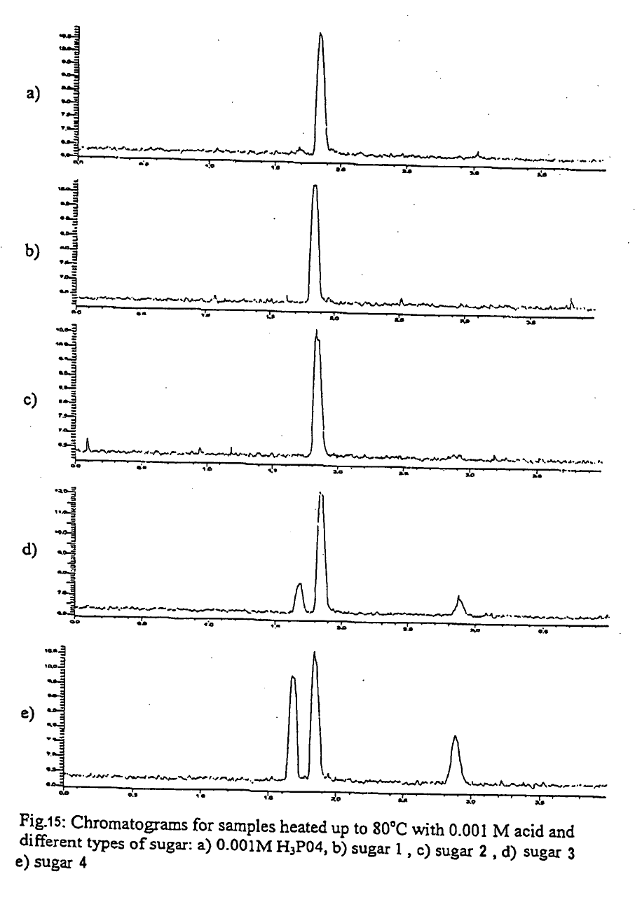

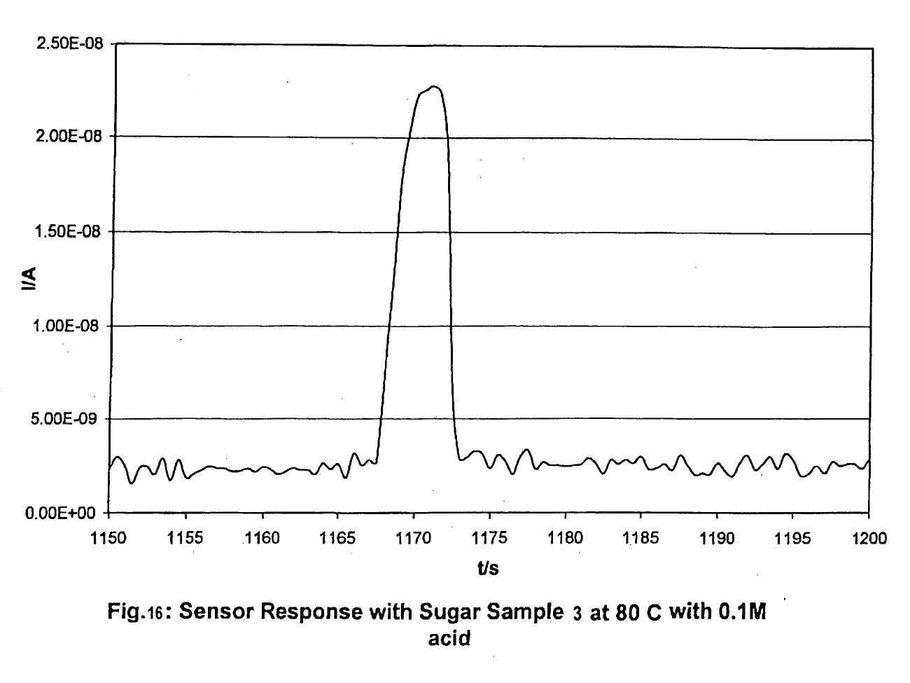

The chromatograms are shown in FIGS. 9 through 15. A typical ECS response is shown in FIG. 16. Calibration measurements showed that a 500 ppb H₂S sample yields an ECS response of 49 nA and a mean GC area count of about 180,000. A 1 ppm sample of SO₂ gives, in agreement with earlier ECS results, an ECS response of 17.5 nA and a mean area count of about 300,000. A 5 ppm COS/100 ppb H₂S/air sample gives a mean area count of about 1,860,000 for COS and 33,000 area counts for H₂S with an ECS signal of 9.8 nA. The ECS response for this gas mix was equivalent to the response of a 100 ppb H₂S fair mix, in agreement with earlier ECS results which yielded no ECS response for COS. The GC peak for H₂S was obtained at 1.68 minutes, and the peak for both SO₂ and COS was obtained at 1.8 mm in the chromatograms.

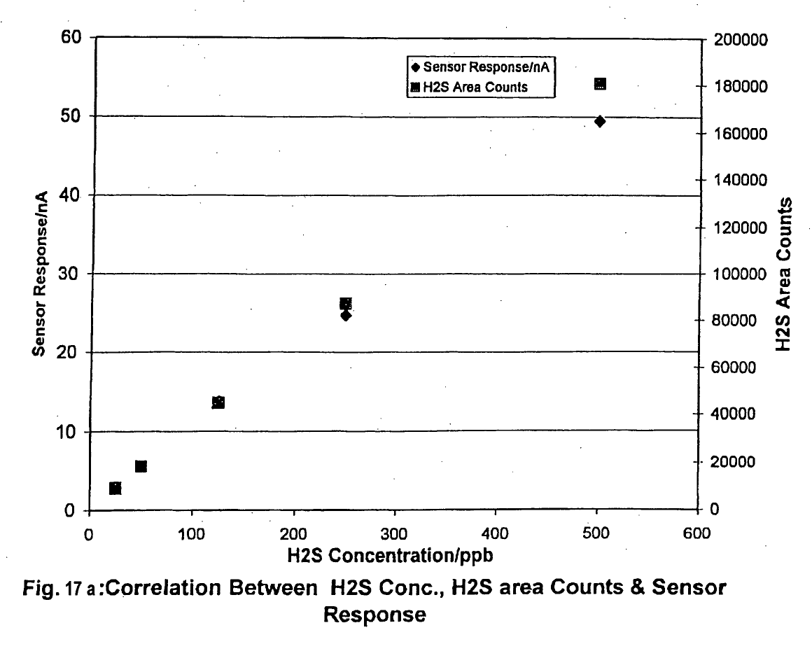

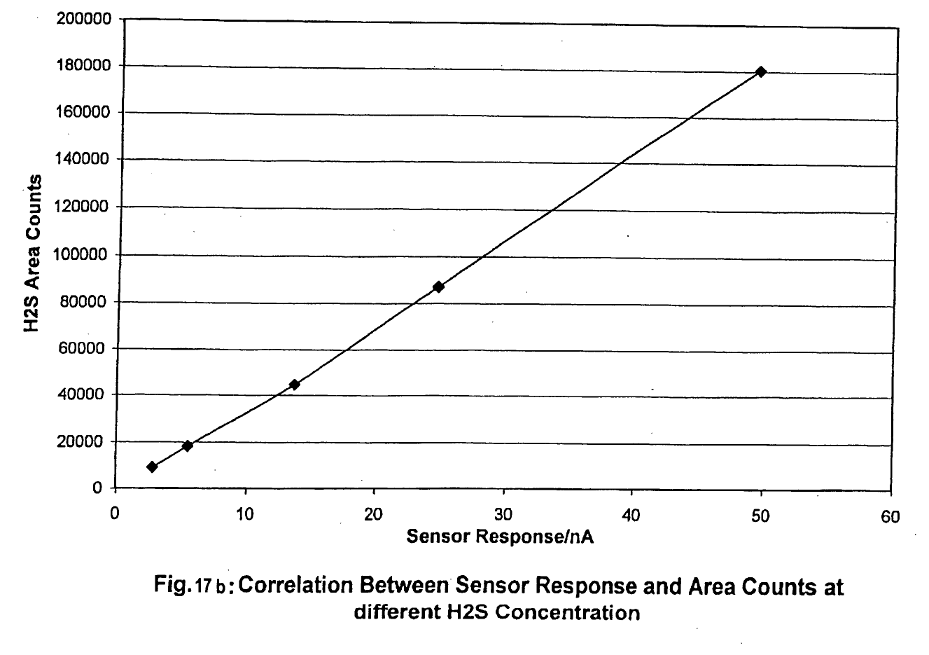

FIGS. 17a and 17b show the linear correlation between the GC area counts and the ECS responses for various H₂S gas concentrations in air as shown in FIG. 7.

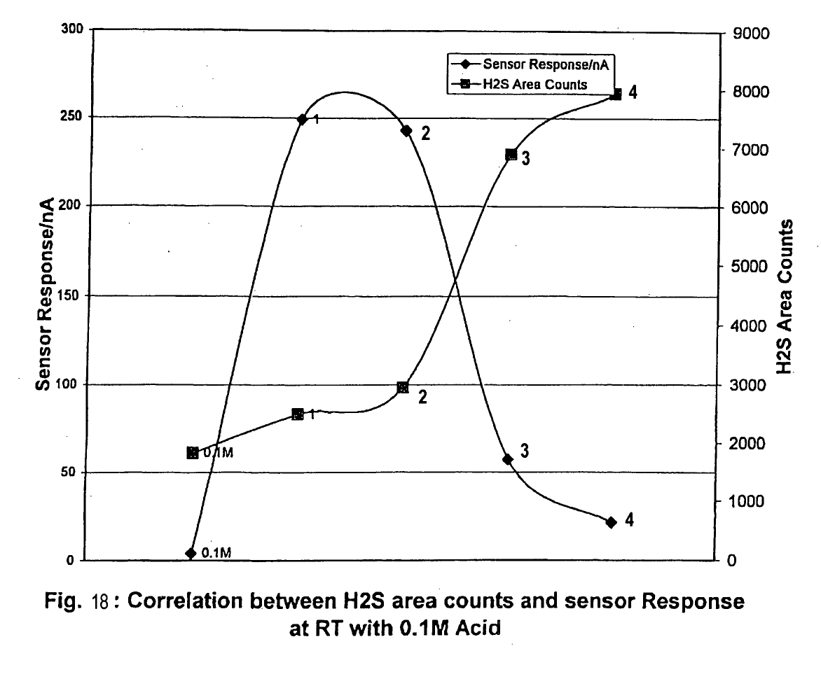

FIGS. 18 through 29 show the correlation between the area counts of H₂S, SO₂, and COS versus the ECS responses in graph form at the various acid concentrations and temperatures, as listed in FIG. 5. The lines connecting the data points are placed for better distinction of the different data groups.

Discussion

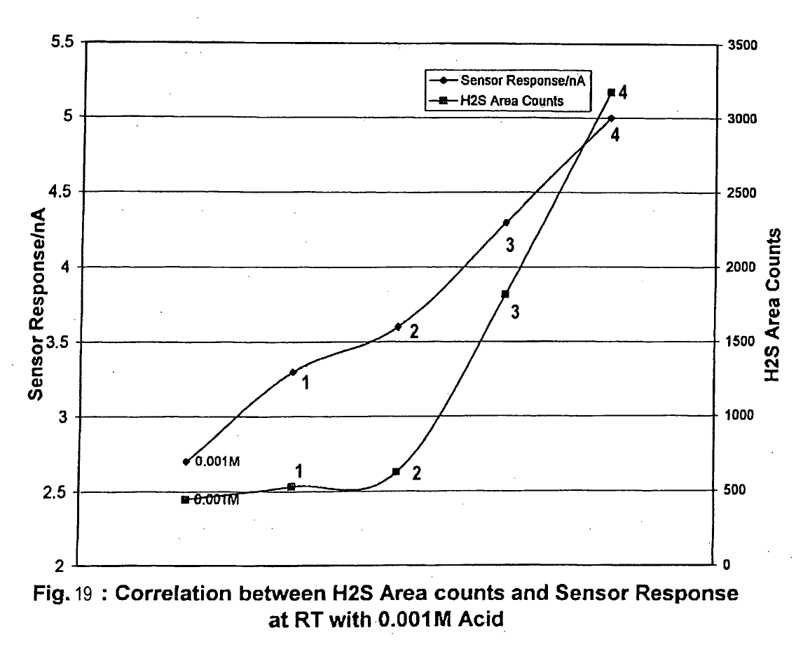

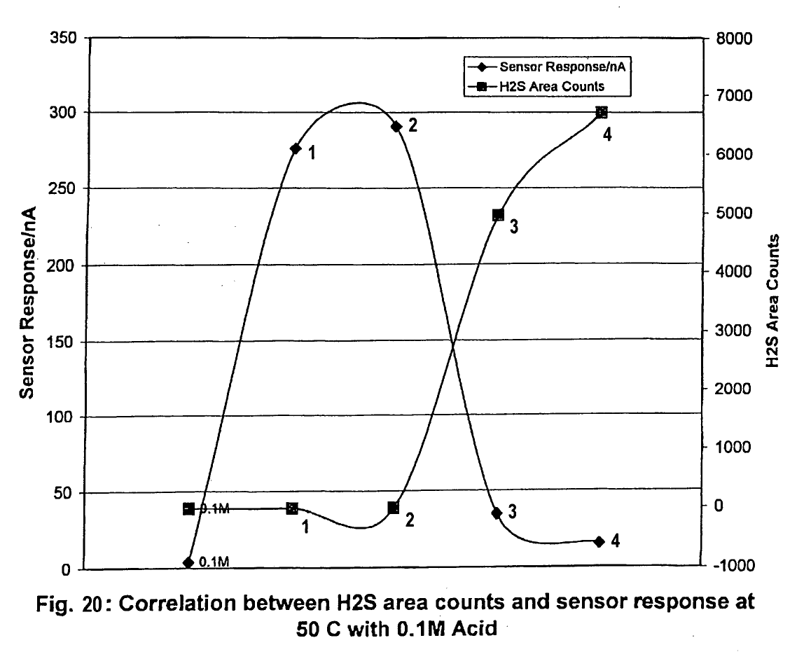

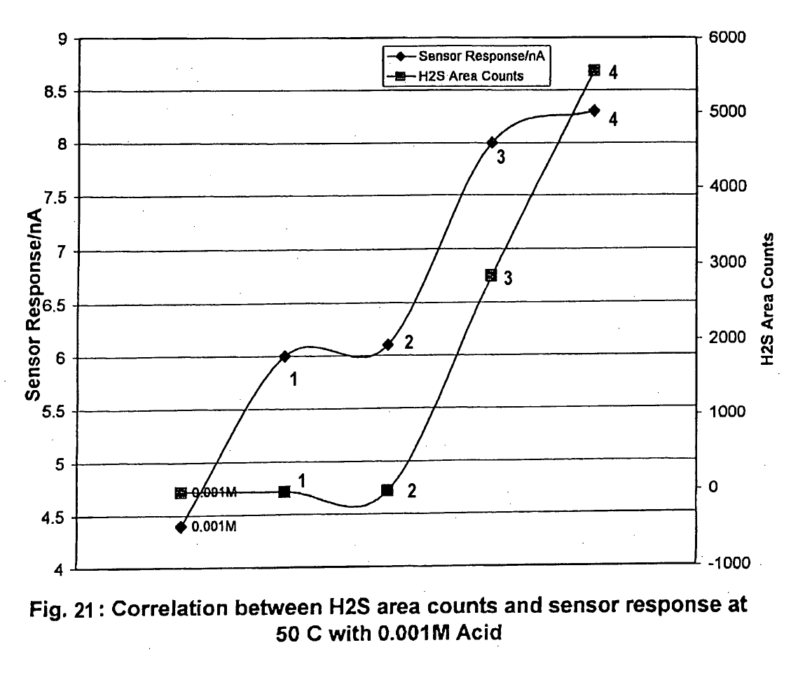

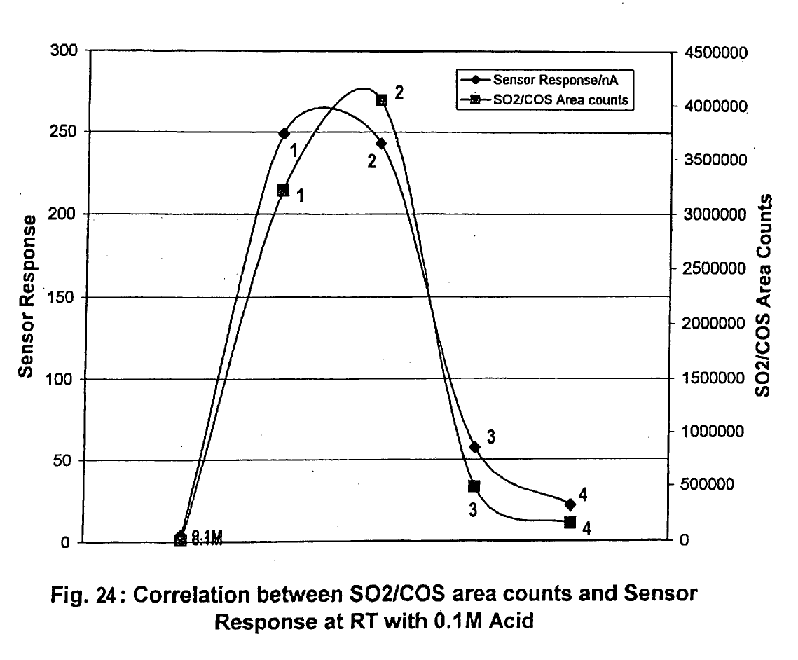

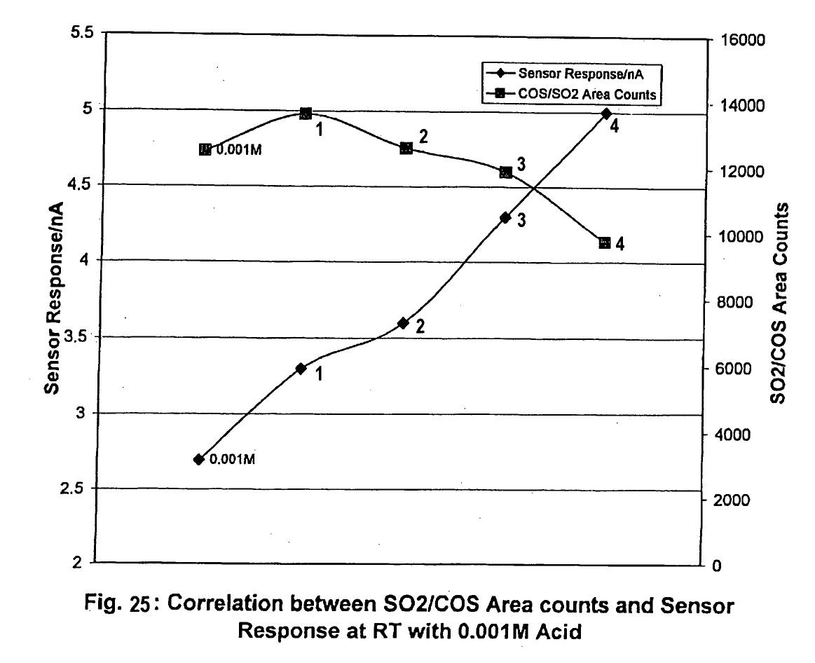

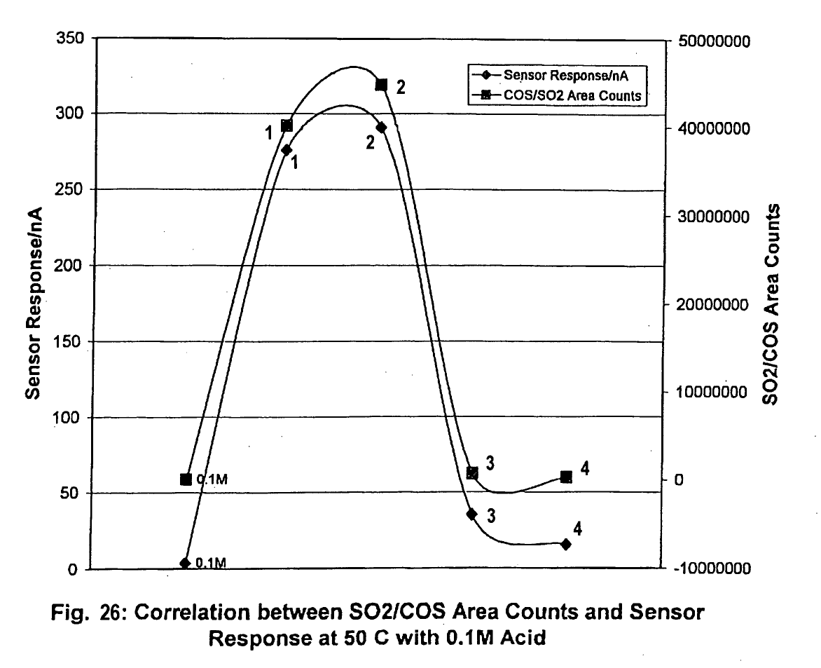

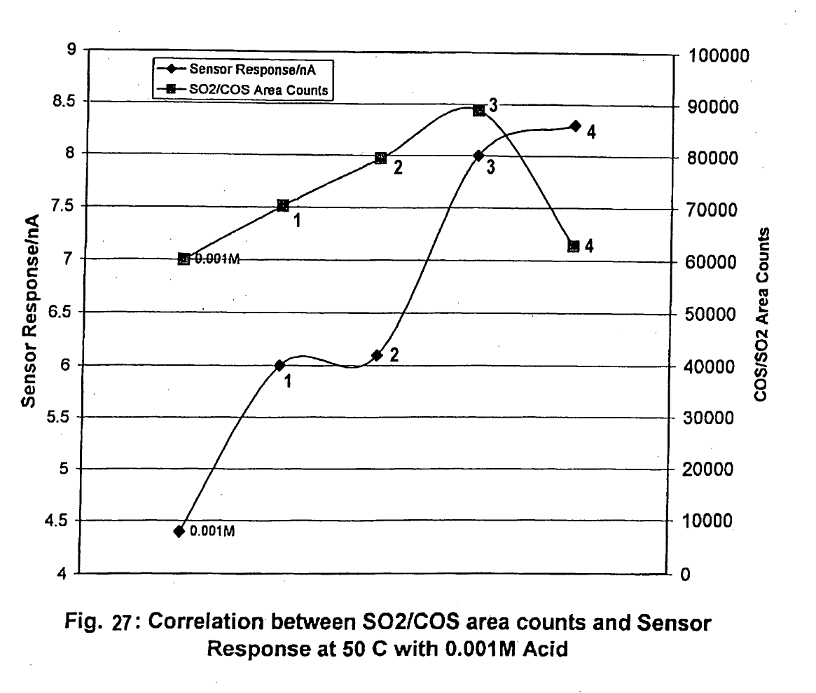

The experiments conducted with 50% sugar/0.1 M acid solution at room temperature (“RT”) and 50° C. showed primarily the presence of SO₂ in the chromatograms (FIGS. 24 and 26). The ECS response for different sugar solutions correlated quite well with the SO₂ peak area counts in chromatograms, while the correlation between the GC and the ECS measurements are poor because of the presence of SO₂ in high (ppm level) concentrations (FIGS. 18 and 20). On the other hand, the experiments conducted with 50% sugar/0.001M acid solutions at RT and 50° C. showed that the formation of SO₂ was suppressed, and peak areas for SO₂/COS were similar to the ones with blank acid solutions. In these cases, the ECS results correlated well with the H₂S peak areas obtained from the sugar solutions (FIGS. 19 and 21). The suppression of SO₂ without significant effect on the release of H₂S can be accomplished by changing the pH of the solution. A gas is released as long as the pH is about one unit or more smaller than the pKA, while the gas stays in the liquid phase when the pH is about one or more units higher than the pKA. The pKA for SO₂ is 1.85 and the pKA for H₂S is 7. Therefore, at a pH of 1 (0.1M acid solution) both H₂S and SO₂ will be released readily, and at pH of 3 (0.001M acid solution) only H₂S will be released into the gas phase (FIGS. 25 and 27).

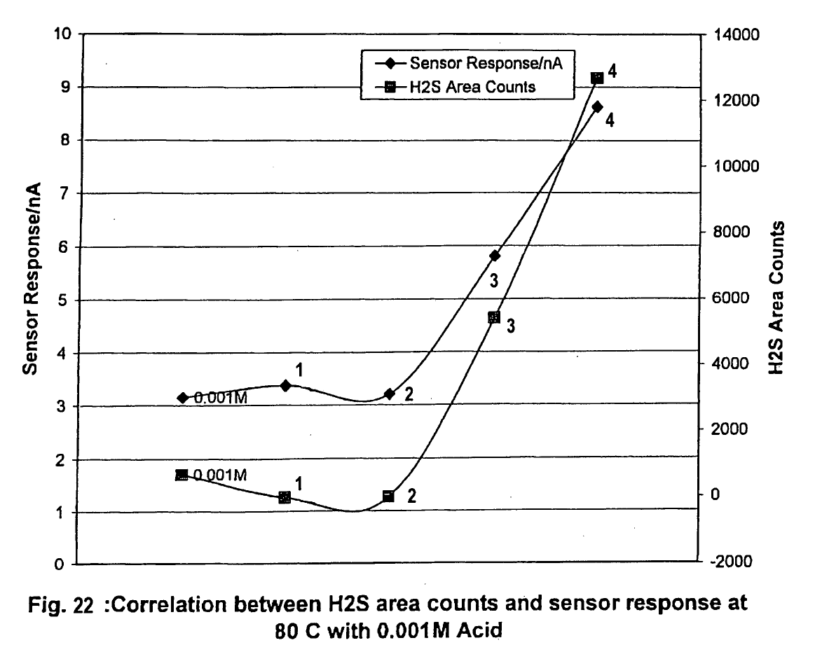

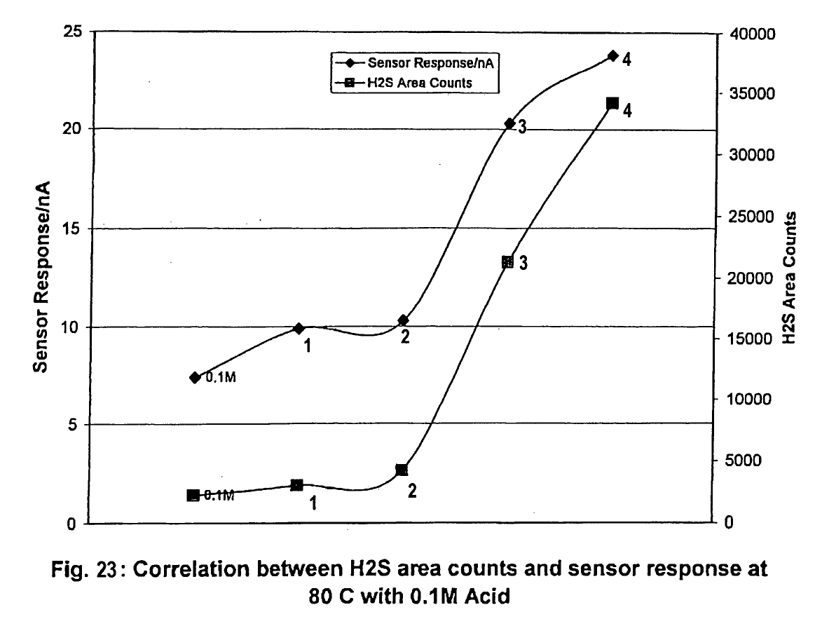

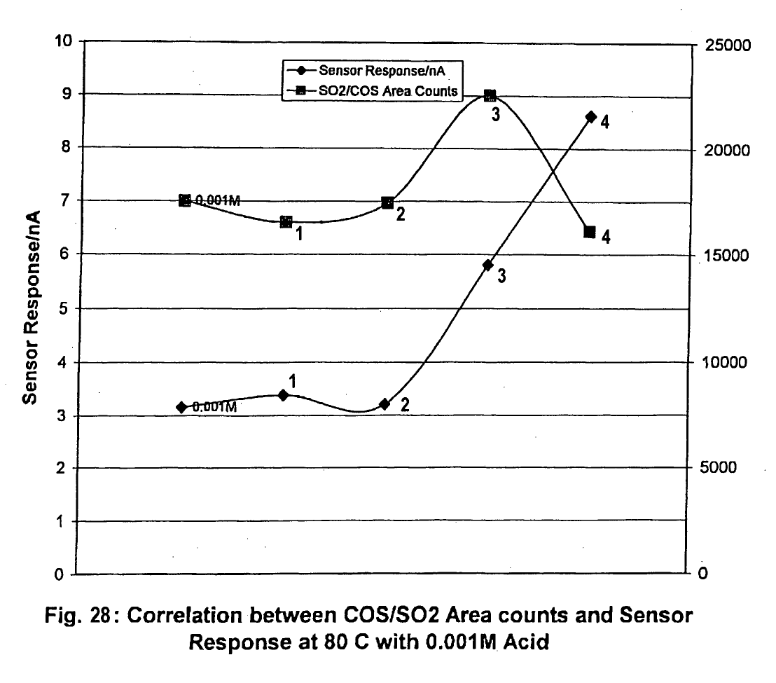

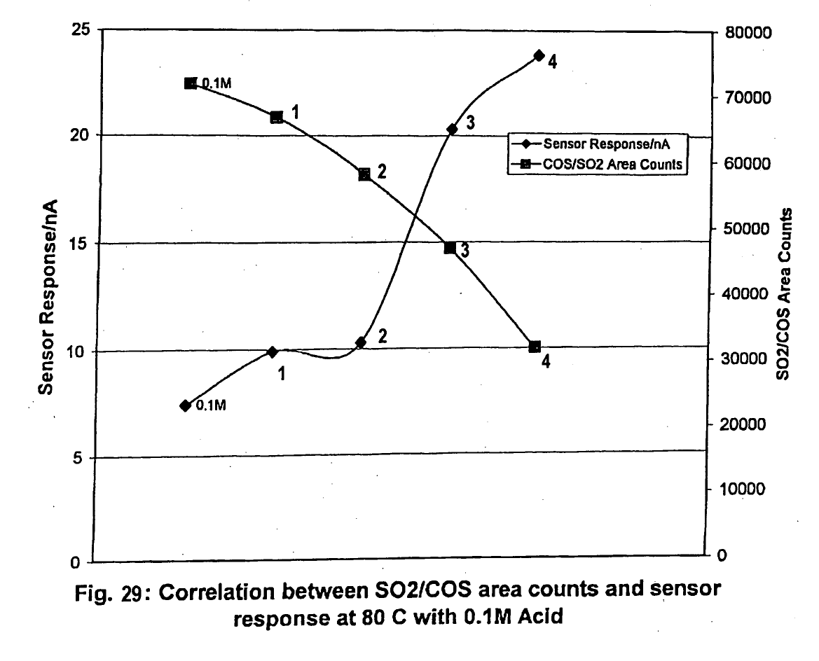

The experiments conducted at 80° C. showed that SO₂/COS peak areas for sugar solutions were significantly reduced and similar in size to blank acid solutions at 0.1M acid concentration due to oxidation of sulfite (which is assumed to be the source of SO₂) to sulfate; thus the correlation between the H₂S area counts and the ECS readings are very good (FIG. 23), while the correlation between SO₂/COS area counts and the ECS readings are poor (FIG. 29). The 0.001M acid solutions showed similar results for SO₂/COS and H₂S peaks and ECS device measurements (FIGS. 22 and 28). Due to the absence of SO₂, the electrochemical sensor results correlate well with H₂S release at 80° C. under different pH conditions.

Among the sugar samples tested, only sugar samples 3 and 4 released H₂S under different pH and temperature conditions. The sugar samples 1 and 2 showed the release of small amounts of H₂S only at a 0.1 M acid concentration and 80° C.

The experiments at different temperatures showed that the released amount of H₂S increased by about a factor of 4 during a temperature increase from RT to 80° C. In addition, it was observed that a change in the heating time of the sugar samples from 30 to 60 minutes showed an increase in the H₂S release by about 25%-30%. At 0.001M acid solutions, the H₂S release was 2-3 times lower than the H₂S release at 0.1 M acid solution.

What is claimed is:

1. A method for determining a concentration of a component in a mixture; comprising:
preparing a reactant having a specified pH level and a specified volume;
combining the mixture with the reactant;
varying the pH level of the combination of the reactant and the mixture;
varying the temperature of the combination of the reactant and the mixture;
oxidizing the combination of the reactant and the mixture;
releasing volatiles from the at least one selected component; and
detecting an indication of a concentration of the at least one selected component based on the released volatiles.

2. The method according to claim 1, further comprising the step of transforming the at least one selected component to a gaseous phase.

3. The method according to claim 1, further comprising the step of determining a concentration of the at least one selected component in the mixture based on the detected indication.

4. The method according to claim 1, further comprising the step of determining a dissociation constant of the at least one selected component and adjusting the pH level of the reactant based on the dissociation constant.

5. The method according to claim 4, further comprising the step of raising the pH level above the dissociation constant to suppress at least one unselected component from releasing volatiles.

6. The method according to claim 4, further comprising the step of lowering the pH level below the dissociation constant to facilitate releasing volatiles from the at least one selected component.

7. The method according to claim 1, further comprising the step of combining the mixture in a basic solution.

8. The method according to claim 1, further comprising the step of combining the mixture in an acidic solution.

9. The method according to claim 1, further comprising the step of suppressing at least one unselected component in the mixture to hinder the at least one unselected component from releasing volatiles.

10. The method according to claim 1, further comprising the step of converting the at least one selected component.

11. A method for determining a concentration of a component in a mixture, comprising:
preparing a reactant having a specified pH level;
combining the mixture with the reactant;
varying the pH level of the combination of the reactant and the mixture to facilitate converting at least one selected component;
releasing volatiles from the least one selected component; and
detecting an indication of a concentration of the at least one selected component based on the released volatiles.

12. The method according to claim 11, further comprising the step of determining a concentration of the at least one selected component in the mixture based on the detected indication.

13. The method according to claim 11, further comprising the step of suppressing at least one unselected component in the mixture to hinder the at least one unselected component from releasing volatiles.

14. A method for determining a concentration of a component in a mixture, comprising:
preparing a reactant having a specified temperature;
combining the mixture with the reactant;
varying the temperature of the combination of the reactant and the mixture to facilitate converting at least one selected component;
releasing volatiles from the least one selected component; and
detecting an indication of a concentration of the at least one selected component based on the released volatiles.

15. The method according to claim 14, further comprising the step of determining a concentration of the at least one selected component in the mixture based on the detected indication.

16. The method according to claim 14, further comprising the step of suppressing at least one unselected component in the mixture to hinder the at least one unselected component from releasing volatiles.

17. A method for determining a concentration of a component in a mixture, comprising:
preparing a reactant having a specified pH level and a specified volume;
combining the mixture with the reactant;
converting the at least one selected component;
releasing volatiles from the at least one selected component; and
detecting an indication of a concentration of the at least one selected component based on the released volatiles.

18. The method according to claim 17, further comprising the step selected from the group consisting of varying the temperature of the combination of the reactant and the mixture, varying the pH level of the combination of the reactant and the mixture, oxidizing the combination of the reactant and the mixture, reducing the combination of the reactant and the mixture, and combinations thereof.

19. The method according to claim 1, further comprising the step of reducing the combination of the reactant and the mixture to facilitate converting the at least one selected component.
